(19) United States
(12) Patent Application Publication
(10) Pub. No.: US 2022/0162601 A1
(43) Pub. Date: May 26, 2022

(54) HIGH THROUGHPUT GENE EDITING SYSTEM AND METHOD

(71) Applicant: Recursion Pharmaceuticals, Inc., Salt Lake City, UT (US)

(72) Inventors: Jacob Cooper, Sandy, UT (US); James Jensen, Centerville, UT (US); Timothy Dahlem, Salt Lake City, UT (US)

(21) Appl. No.: 17/101,545

(22) Filed: Nov. 23, 2020

(51) Int. Cl. C12N 15/113; C12N 9/22; B01L 3/00

(52) U.S. Cl. CPC C12N 15/113; C12N 9/22; B01L 2300/0829; C12N 2310/20; C12Y 301/00; B01L 3/5085

(57) ABSTRACT

The present disclosure provides CRISPC-Cas systems for the creating mutated genes within cells within wells of an addressable well plate.

### FIELD

The present disclosure generally relates to high throughput methods of creating genetic mutations in cells using CRISPR reagents, lipofection, and addressable array multi-well plates.

### BACKGROUND

Methods of using CRISPR reagents to create genetic mutations are known. See WO2018/057837, US2019/0233820, WO2019/025984, WO2015/089465, US2019/0153412, and WO2015/089473. However, methods of handling CRISPR reagents to improve introduction into cells and increase CRISPR activity on a high throughput platform are needed.

### SUMMARY

Aspects of the present disclosure are directed to compositions and methods of multiplex gene editing using a CRISPR/Cas system including a Cas enzyme and a guide RNA as is known in the art to edit a target gene in a target cell present in each of a plurality of wells in a well plate. Each well includes a guide RNA for a different target gene of the target cell. Accordingly, each well contains a CRISPR/Cas system that targets a single genetic locus for mutation. When considering each different single genetic locus targeted for mutation in each well, the genome of the target cell is effectively subject to multiplex gene editing in parallel since the method results in multiple genes of the target cell, as a whole, being edited. In this manner, a method of high throughput screening is provided whereby a CRISPR-Cas system is used to create a single mutated gene within each well of an addressable or indexable multi-well plate.

According to one aspect, the system includes (1) a liposome comprising a guide RNA, wherein the guide RNA includes a unique spacer complementary to a target gene, (2) a liposome comprising a Cas enzyme or a nucleic acid sequence encoding the Cas enzyme, and (3) a target cell. The guide RNA may be a dual guide RNA, i.e. including a separate crRNA molecule and a separate tracrRNA molecule that hybridize together, as is known in the art, or the guide RNA may be a single guide RNA, i.e. a single molecule having a crRNA portion and a tracrRNA portion linked together, i.e. covalently linked together, by a linker, such as a nucleic acid sequence, as is known in the art. Linkers, such as GAAA, are known to those of skill in the art. In a general aspect, (1) the liposome comprising the guide RNA, (2) the liposome comprising a Cas enzyme or a nucleic acid sequence encoding the Cas enzyme, and (3) the target cells are present within a well of the well plate. The Cas enzyme and the guide RNA form a colocalization complex with the target gene of the target cell and the Cas enzyme generates a double stranded break in the target gene which induces one or more mutations in the gene when the double stranded break is repaired by the cell.

According to one aspect, the system includes (1) a liposome comprising a crRNA portion of a guide RNA, wherein the crRNA portion includes a unique spacer complementary to a target gene and a segment complementary to a segment of a tracrRNA, (2) a liposome comprising a tracrRNA portion of the guide RNA, wherein the tracrRNA portion hybridizes to the crRNA portion, (3) a liposome comprising a Cas enzyme or a nucleic acid sequence encoding the Cas enzyme, and (4) a target cell (which may be a plurality of the same cell type within a given well). In a general aspect, (1) the liposome comprising a crRNA portion of a guide RNA, (2) the liposome comprising a tracrRNA portion of the guide RNA, (3) the liposome comprising a Cas enzyme or a nucleic acid sequence encoding the Cas enzyme, and (4) the target cell are present within a well of the well plate. The crRNA portion and the tracrRNA portion hybridize to form the guide RNA. The Cas enzyme and the guide RNA form a colocalization complex with the target gene of the target cell and the Cas enzyme generates a double stranded break in the target gene which induces one or more mutations in the gene when the double stranded break is repaired by the cell.

According to one aspect, methods described herein are carried out in a plurality of wells in the well plate wherein the guide RNA in each well targets a different target gene. According to one aspect, this method is carried out simultaneously in a plurality of wells in the well plate wherein the guide RNA in each well targets a different target gene.

According to one aspect, (1) the liposome comprising a crRNA portion of a guide RNA, (2) the liposome comprising a tracrRNA portion of the guide RNA, (3) the liposome comprising a Cas enzyme or a nucleic acid sequence encoding the Cas enzyme, and (4) the target cell may be introduced into the well simultaneously. According to one aspect, (1) the liposome comprising a crRNA portion of a guide RNA, (2) the liposome comprising a tracrRNA portion of the guide RNA, (3) the liposome comprising a Cas enzyme or a nucleic acid sequence encoding the Cas enzyme, and (4) the target cell may be introduced into the well in series or a combination of one or more, two or more or three or more of (1), (2), (3) or (4) may be introduced into the well simultaneously. For example, the (4) target cell may be introduced into the well, and then a mixture of the (1) the liposome comprising a crRNA portion of a guide RNA, (2) the liposome comprising a tracrRNA portion of the guide RNA, and (3) the liposome comprising a Cas enzyme or a nucleic acid sequence encoding the Cas enzyme may be introduced into the cell. For example, a mixture of the (4) target cell, (2) the liposome comprising a tracrRNA portion of the guide RNA, and (3) the liposome comprising a Cas enzyme or a nucleic acid sequence encoding the Cas enzyme may be introduced into the cell, and then the (1) the liposome comprising a crRNA portion of a guide RNA may be introduced into the cell. Other embodiments of introducing (1) the liposome comprising a crRNA portion of a guide RNA, (2) the liposome comprising a tracrRNA portion of the guide RNA, (3) the liposome comprising a Cas enzyme or a nucleic acid sequence encoding the Cas enzyme, and (4) the target cell are envisioned and described further herein. In addition, embodiments of introducing (1) the liposome comprising a guide RNA, (2) the liposome comprising a Cas enzyme or a nucleic acid sequence encoding the Cas enzyme, and (3) the target cell simultaneously or separately or as a mixture of (1) and (2), or (2) and (3), or (1) and (3) are envisioned.

According to another aspect, the present disclosure provides a nucleic acid encoding the Cas enzyme. In some embodiments, the nucleic acid encoding the Cas enzyme is present in a vector, such as an engineered DNA plasmid vector or a viral vector. The nucleic acid includes a promoter for expression of the Cas enzyme within the target cell. According to one aspect, the Cas enzyme is provided to the target cell by introduction of the plasmid into the target cell and expression of the Cas enzyme. According to one aspect, the Cas enzyme may have one or more or two or more nuclear localization signals to facilitate the Cas enzyme entering the nucleus of the target cell.

According to one aspect, target cells within wells of a well plate, wherein the target cells are of the same species type for example, are combined with CRISPR-Cas system components within liposomes which facilitate entry of the CRISPR-Cas components, i.e. guide RNA and Cas enzyme into the target cells to carry out gene cutting and creation of mutated genes. The cells with the mutated genes are then analyzed. According to one aspect, the target cell is a eukaryotic cell, such as a yeast cell, a plant cell, a mammalian cell or a human cell. According to one aspect, the cell is a prokaryotic cell. According to one aspect, the well plate is an addressable or indexable or identifiable array insofar as the identity and location of the guide RNA, such as having a unique spacer sequence, within each well within the array is known. Accordingly, the identity and location of the target gene to be mutated based on the identity of the spacer sequence, is known within the array. The process may be repeated in a subsequent addressable array well plate where each guide RNA is provided to a well location that is different from the well location of a prior well plate. The method can be repeated in an addressable array well plate a plurality of times with the guide RNA being provided at different locations of the addressable array well plate.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts in schematic one embodiment of the present disclosure.

FIG. 2 depicts in schematic an alternate embodiment of the present disclosure.

FIG. 3 depicts in schematic an alternate embodiment of the present disclosure.

FIG. 4 depicts in schematic an alternate embodiment of the present disclosure.

FIG. 5 depicts gene editing rate data of cells targeted for specific gene knockout.

FIG. 6 depicts mitochondria morphology.

FIG. 7 depicts distribution of cell counts.

### DETAILED DESCRIPTION

According to aspects of the present disclosure, CRISPR protein endonucleases, such as Cas9, are provided, which comprise at least one nuclear localization signal, at least one nuclease domain, and at least one domain that interacts with a guide RNA to target the endonuclease to a specific nucleotide sequence for cleavage. Also provided are nucleic acids encoding CRISPR protein endonucleases, as well as methods of using CRISPR protein endonucleases to modify chromosomal sequences of cells such as eukaryotic cells or cells of embryos. CRISPR protein endonucleases interact with specific guide RNAs, each of which directs the endonuclease to a specific targeted site, at which site the CRISPR protein endonucleases introduces a double- stranded break that can be repaired by a DNA repair process such that the chromosomal sequence is modified or mutated. Since the specificity is provided by the guide RNA (or the crRNA), the CRISPR protein endonucleases are universal and can be used with different guide RNAs to target different genomic sequences. Methods disclosed herein can be used to target and modify specific chromosomal sequences at targeted locations in the genome of cells.

The methods described herein using CRISPR-Cas systems provide a platform for high efficiency genome editing of cells. Cells and the CRISPR-Cas system components are delivered to wells of a multi-well plate where the CRISPR-Cas system components are delivered to the cell using liposome-mediated transfection, for example, and are used to cut a target nucleic acid in the genome of the cell. The use of a multi-well plate and CRISPR-Cas system components produces high throughput, multiplexed genome-wide cell editing.

Aspects of the present disclosure are directed to methods of mutating a target gene within a target cell within a well of an addressable array well plate. A plurality of target cells is within the addressable array well plate. The plurality of cells is contacted with a CRISPR-Cas system, components of which are present within one or more liposomes for entry into the target cells. Exemplary CRISPR-Cas systems include a guide RNA and a Cas enzyme, such as Cas9, and are known to those of skill in the art and are commercially available. The spacer sequence of a guide RNA can be designed as is generally known in the art to target a target gene. Guide RNAs with designed spacer sequences are commercially available. The CRISPR-Cas system components enter the cell and target genes are mutated in the plurality of target cells. The well plate is addressable insofar as the identity of a guide RNA spacer sequence, and thus the target gene, is at a known location within the well plate. According to one aspect, each cell of the plurality of cells is of the same species type. According to one aspect, each cell of the plurality of cells includes a different mutated gene. Accordingly, a plurality of genes of the target cell are mutated. Collectively, the plurality of mutated genes represents multiplex mutation of the target cell and analysis thereof provides a high throughput screening method. According to one aspect, the disclosure includes the use of multi-well plates, as well as high throughput methods employing such plates, in which different wells contain Cas9 protein and a transfection reagent. Further, different wells contain different gRNA molecules. Such plates may be used in high throughput methods for altering multiple genetic sites within cells.

According to one aspect, the disclosure provides ready to use reagents. For example, a ready to use reagent may include a mixture of a Cas protein, such as Cas9, or nucleic acid encoding a Cas protein and a tracrRNA portion of a guide RNA or a nucleic acid encoding a tracrRNA portion of a guide RNA. The mixture may then be combined with a cell and a crRNA or nucleic acid encoding a crRNA resulting in CRISPR activity.

### Crispr Systems

Embodiments of the present disclosure are directed to CRISPR/Cas based systems, components of which may be within liposomes to facilitate entry into a target cell. CRISPR systems, i.e. those including a Cas enzyme and a guide RNA, having nuclease activity are known to those of skill in the art. Such Cas enzymes include Cas enzymes of a Type II CRISPR system, i.e. Cas enzymes that function as part of a Type II CRISPR system. Such Cas enzymes include naturally occurring or wild type Cas enzymes, for example, in Type II CRISPR systems or mutants or modifications thereof. Cas9 is an exemplary Cas enzyme. Cas enzymes are commercially available as described herein and may be modified from their wild type version, as described in Vakulskas et al., Nature Medicine, vol. 24, Aug. 2018, pp. 1216-1224 hereby incorporated by reference in its entirety describing an exemplary single point mutation R691A in Cas9. For example, Cas9 as used herein is commercially available from Integrated DNA Technologies, Inc., Coralville, Iowa, USA. Exemplary Cas enzyme nickases are known in the art which include one or more point mutations. Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. Useful Cas enzymes, such as commercially available Cas9, include those having at least 99%, 98%, 97%, 96%, 95%, 90%, 85%, 80%, 75% or 70% sequence identity or homology to the wild-type Cas enzyme (such as Cas9) as described in the art, such as Vakulskas et al., Nature Medicine, vol. 24, Aug. 2018, pp. 1216-1224, while retaining their enzymatic function to create a double stranded break in a target nucleic acid. Methods of determining sequence identity are known to those of skill in the art. According to one aspect, the Cas enzyme may be provided in a liposome.

As used herein the term “CRISPR activity” refers to an activity associated with a CRISPR system. Examples of such activities are cutting or nicking of a target nucleic acid. As used herein the term “CRISPR system” refers to a collection of CRISPR proteins and nucleic acid that, when combined, result in at least CRISPR associated activity (e.g., the target locus specific, double-stranded cleavage of double-stranded DNA). As used herein the term “CRISPR complex” refers to the CRISPR proteins and nucleic acid (e.g., guide RNA) that associate with each other to form an aggregate that has functional activity. CRISPR proteins include those that are wild-type or modifications or mutants thereof and may be commercially available in either wild-type or modified or mutant form. An example of a CRISPR complex is Cas9 (sometimes referred to as Csn1) protein that is bound to a guide RNA specific for a target locus. In many instances, CRISPR proteins will contain nuclear localization signals (NLS) that allow them to be transported to the nucleus. As used herein the term “target locus” refers to a site within a nucleic acid molecule for CRISPR system interaction (e.g., binding and cleavage). When a single CRISPR complex is designed to cleave double-stranded nucleic acid, then the target locus is the cut site and the surrounding region recognized by the CRISPR complex. When two CRISPR complexes are designed to nick double-stranded nucleic acid in close proximity to create a double-stranded break, then the region surrounding and including the break point is referred to as the target locus.

In general, CRISPR-Cas systems described herein rely on a guide RNA in complex with a Cas protein to target a nucleic acid sequence. See US 2018/0195089 hereby incorporated by reference in its entirety for description of CRISPR-Cas systems and methods of introducing CRISPR/Cas systems into cells. CRISPR systems do not require the generation of customized proteins to target specific sequences but rather a single Cas enzyme that can be directed to a target nucleotide sequence (a target locus) by a short RNA molecule with sequence complementarity to the target.

In general, three classes of CRISPR systems are generally known and are referred to as Type I, Type II or Type III. According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June 2011) hereby incorporated by reference in its entirety. According to one aspect, the Cas9 unwinds the DNA duplex and searches for sequences matching the crRNA to cleave. Target recognition occurs upon detection of complementarity between a “protospacer” sequence in the target DNA and the remaining spacer sequence in the crRNA. Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January 2011) each of which are hereby incorporated by reference in their entireties.

Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csyl, Csy2, Csy3, Csel (or CasA), Cse2 (or CasB), Cse3 (or CasE), Cse4 (or CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Cszl, Csx15, Csf1, Csf2, Csf3, Csf4, and Cu1966. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua;Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alphal4; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

CRISPR systems and delivery methods to cells useful in the present disclosure are also described in WO2018/057837, US2019/0233820, WO2019/25984, WO2015/089465 US2019/0153412, WO2015/089473, WO2018/094356 and US2019/0390229 each of which are hereby incorporated by reference in their entireties.

According to one aspect, a CRISPR protein having two or more nuclease domains may be modified or altered to inactivate all but one of the nuclease domains. Such a modified or altered CRISPR protein is referred to as a nickase, to the extent that the CRISPR protein cuts or nicks only one strand of double stranded DNA. An exemplary CRISPR protein is of a Type II CRISPR System, such as a Cas9 protein or modified Cas9 or homolog of Cas9 or mutant of Cas9. An exemplary Cas9 protein nickase is known in the art. See Jinek et al., Science 337, 816-821 (2012). According to one aspect, the Cas9 protein or Cas9 protein nickase includes homologs and orthologs thereof which retain the ability of the protein to bind to the DNA and be guided by the RNA. According to one aspect, the Cas9 protein includes the sequence as set forth for naturally occurring Cas9 from S. thermophiles, S. aureus or S. pyogenes and protein sequences having at least 20%, 30%, 40%, 50%, 60%, 70%, 75% 80%, 85%, 90%, 95%, 96%, 97%, 98% or 99% homology thereto.

An exemplary CRISPR system includes the S. thermophiles or S. aureus Cas9 nuclease (ST1 Cas9, Sa Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S.H., Redding, S., Jinek, M., Greene, E.C. & Doudna, J.A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to one aspect, the Cas9 protein is an enzymatically active Cas9 protein, a Cas9 protein wild-type protein, or a Cas9 protein nickase.

According to certain aspects, a Cas9 protein, for example, may be either introduced into cells or produced intracellularly. Further, the duration of time that Cas9 protein is taken up or produced intracellularly and the amount that is present intracellularly may be controlled or regulated. As an example, a chromosomally integrated Cas9 protein coding sequence may be operably linked to a regulatable promoter. Further, the amount of mRNA encoding Cas9 protein introduced into cells may be regulated.

### Guide RNA

Embodiments of the present disclosure are directed to the use of a CRISPR/Cas system and, in particular, a guide RNA which may include one or more of a spacer sequence, a tracr mate sequence and a tracr sequence. The guide RNA may be provided in a liposome. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. Guide RNA molecules may have a region of sequence complementarity (known in the art as a spacer) of at least 10 (e.g., from about 10 to about 50, from about 10 to about 40, from about 10 to about 35, from about 10 to about 30, from about 10 to about 25, from about 15 to about 25, from about 17 to about 22, of about 20, etc.) nucleotides to a target locus. In many instances, the target locus is a naturally occurring chromosomal locus in a eukaryotic cell. Spacer sequences can be designed, such as by using bioinformatic data, to target a nucleic acid sequence within a genome of a cell. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. Accordingly, the crRNA portion of a guide RNA and the tracrRNA portion of the guide RNA are linked and provided as a single guide RNA within a liposome. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA). When a guide RNA is a two component species, guide RNA molecules with specificity for different target sites can be generated using a single tracrRNA molecule/segment connected to a target site specific crRNA molecule/segment. For example, different crRNA molecules/segments can be designed that target different nucleic acids within a genome and the single tracrRNA molecule/segment can hybridize to each of the different crRNA molecules/segments to form functional guide RNA targeting different nucleic acids within the genome. According to this aspect, the tracrRNA molecule can be introduced into each well along with the cell and the Cas protein, as a mixture for example. Then, a different target specific crRNA can be added to each well, thereby creating a different genetic mutation of the cell genome within each well.

Guide RNA molecules can be designed and synthesized using methods known to those of skill in the art. For example, target nucleic acid sequences suitable for CRISPR editing (known as protospacer sequences) may be identified in the genome of a cell. Complementary sequences (known as spacer sequences) can be synthesized. The spacer sequences can be connected to a scaffold sequence known to those of skill in the art to bind to a Cas protein. In this manner, a functional guide RNA including crRNA segment and a tracrRNA segment can be created. As an example, a spacer sequence (about 15 to 30 nucleotides) is the target locus recognition sequence of a crRNA including a portion (about 10-30 nucleotides) to hybridize to the tracrRNA. The tracrRNA (including 10-50 nucleotides) hybridizes to the crRNA to form the guide RNA. The crRNA may be linked to the tracrRNA via a linker nucleic acid segment.

According to one aspect, collections of crRNA molecules are provided with specificity for individual target sites. For example, collections of crRNA molecules with specificity for target sites within particular types of cell (e.g., human cells) and provided. The members of such collection of cells may be generated based upon sequence information for these particular types of cells. As an example, one such collection could be generated using the complete genome sequence of a particular type of cell. The genome sequence data can be used to generate a library of crRNA molecules with specificity for the coding region of each gene within the human genome. Parameters that could be used to generate such a library may include the location of protospacer adjacent motif (PAM) sites, off target effects (e.g., sequences unique to the target region), and, when gene “knockouts” are desired, locations within coding regions likely to render the gene expression product fully or partially non-functional (e.g., active site coding regions, intron/exon junctions, etc.). Collections or libraries of crRNA molecules may include a wide variety of individual molecules such as from about five to about 100,000 e.g., from about 50 to about 100,000, from about 200 to about 100,000, from about 500 to about 100,000, from about 800 to about 100,000, from about 1,000 to about 100,000, from about 2,000 to about 100,000, from about 4,000 to about 100,000, from about 5,000 to about 100,000, from about 50 to about 50,000, from about 100 to about 50,000, from about 500 to about 50,000, from about 1,000 to about 50,000, from about 2,000 to about 50,000, from about 4,000 to about 50,000, from about 50 to about 10,000, from about 100 to about 10,000, from about 200 to about 10,000, from about 500 to about 10,000, from about 1,000 to about 10,000, from about 2,000 to about 10,000, from about 4,000 to about 10,000, from about 50 to about 5,000, from about 100 to about 5,000, from about 500 to about 5,000, from about 1,000 to about 5,000, from about 50 to about 2,000, from about 100 to about 2,000, from about 500 to about 2,000, etc. The number of crRNA molecules may be determined by the number of genes to be mutated for a given genome of a given target cell.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction. In exemplary embodiments, guide RNA coding sequence is packaged into liposomes and delivered to cells. According to one aspect, a different crRNA is delivered to each well of a plurality of wells in order to create a different genomic mutation in each well.

### Cells

Cells according to the present disclosure include any cell into which the disclosed nucleic acids can be introduced and expressed as described herein. It is to be understood that the basic concepts of the present disclosure described herein are not limited by cell type. Representative cells that may be used in the practice of the invention include, but are not limited to, bacterial cells, yeast cells, plant cells and animal cells, such as mammalian cells and human cells. In some embodiments, the cell is from an embryo. The cell can be a stem cell, zygote, or a germ line cell. In embodiments where the cell is a stem cell, the stem cell is an embryonic stem cell or pluripotent stem cell. In other embodiments, the cell is a somatic cell. In embodiments, where the cell is a somatic cell, the somatic cell is a eukaryotic cell or prokaryotic cell. The eukaryotic cell can be an animal cell, such as from a pig, mouse, rat, rabbit, dog, horse, cow, non-human primate, or a human cell.

The invention further includes cells containing one or more CRISPR system components and cells made by methods set out herein. For example, the invention includes cells into which CRISPR complexes have been introduced (e.g., cells that contain (1) plasmids encoding Cas9 and guide RNA, (2) Cas9 mRNA and guide RNA, etc.). The invention further includes that have been modified by methods of the invention (e.g., cells that have undergone cleavage and relegation of cellular DNA with and without inserts at the cleavage site) that either contain or no longer contain one or more CRISPR system component.

### Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue- specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)], the SV40 promoter, the dihydrofolate reductase promoter, the (3-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

### Well Plates

Well plates useful in the methods described herein include a plurality of wells as is known in the art. A well plate is generally a flat plate with multiple “wells” into which reagents are placed. The well acts as a test tube or containment for carrying out genome editing using the CRISPR/Cas system. A well plate may have 6, 12, 24, 48, 96, 384, 1536, 3456 or 9600 wells arranged in a rectangular matrix forming an array which may be an addressable array. Each well may hold between tens of nanoliters to several milliliters of liquid. The geometry of the wells can vary from circular to square. Well plates useful in the present disclosure are commercially available.

According to certain aspects, the multi-well plate includes 500 or more wells, 1000 or more wells, 3000 or more wells. According to one aspect, well plate includes edge wells and wherein the edge wells are vacant or are not used during the high throughput methods described herein.

Introduction of Crispr Systems into Cells

According to certain aspects, components of the CRISPR/Cas system or nucleic acids encoding components of the CRISPR/Cas system or vectors including nucleic acids encoding components of the CRISPR/Cas system may be delivered to the cell by methods known to those of skill in the art. Compositions and methods for introduction of CRISPR system components into cells are provided including those described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbour Laboratory Press, Cold Spring Harbour. N.Y. (1989), and include, calcium phosphate transfection, DEAE-dextran mediated transfection, transfection, microinjection, cationic lipid-mediated transfection, electroporation, nucleofection, transduction, scrape loading, ballistic introduction, nucleoporation, hydrodynamic shock, and infection.

Transfection agents suitable for use with the invention include transfection agents that facilitate the introduction of RNA, DNA and proteins into cells. Exemplary transfection reagents include TurboFect Transfection Reagent (Thermo Fisher Scientific), Lipofectamine CRISPRMAX (Thermo Fisher Scientific), Pro-Ject Reagent (Thermo Fisher Scientific), TRANSPASS P Protein Transfection Reagent (New England Biolabs), CHARIOT Protein Delivery Reagent (Active Motif), PROTEOJUICE Protein Transfection Reagent (EMD Millipore), 293fectin, LIPOFECTAMINE 2000, LIPOFECTAMINE 3000 (Thermo Fisher Scientific), LIPOFECTAMINE (Thermo Fisher Scientific), LIPOFECTIN (Thermo Fisher Scientific), DMRIE-C, CELLFECTIN (Thermo Fisher Scientific), OLIGOFECTAMINE (Thermo Fisher Scientific), LIPOFECTACE, FUGENE (Roche, Basel, Switzerland), FUGENE HD (Roche), TRANSFECTAM (Transfectam, Promega, Madison, Wis.), TFX-10 (Promega), TFX-20. (Promega), TFX-50 (Promega), TRANSFECTIN (BioRad, Hercules, Calif.), SILENTFECT (Bio- Rad), EFFECTENE (Qiagen, Valencia, Calif.), DC-chol (Avanti Polar Lipids), GENEPORTER (Gene Therapy Systems, San Diego, Calif.), DHARMAFECT 1 (Dharmacon, Lafayette, Colo.), DHARMAFECT 2 (Dharmacon), DHARMAFECT 3 (Dharmacon), DHARMAFECT 4 (Dharmacon), ESCORT III (Sigma, St. Louis, Mo.), and ESCORT IV (Sigma Chemical Co.).

According to one aspect, lipofection is a lipid-based transfection technology where a cationic lipid is mixed with material to be introduced into a cell to produce liposomes that fuse with the cell membrane and deposit the material inside of the cell. Reagents to create liposomes for lipofection are commercially available and include LIPOFECTAMINE RNAIMAX and LIPOFECTAMINE CRISPRMAX commercially available from ThermoFisher Scientific.

Conditions will normally be adjusted on, for example, per cell type basis for a desired level of CRISPR system component introduction into the cells. Any number of conditions may be altered to enhance the introduction of CRISPR system components into cells. Exemplary incubation conditions are pH, ionic strength, cell type, energy charge of the cells, the specific CRISPR system components present, the ratio of CRISPR system components (when more than one CRISPR system component is present), the CRISPR system component/cell ratio, concentration of cells and CRISPR system components, incubation times, etc.

According to one aspect, the CRISPR components as described herein are provided to a well containing a cell and are then incubated or cultured for a period of time (e.g., from about 2 minutes to about 8 hours, from about 10 minutes to about 8 hours, from about 20 minutes to about 8 hours, from about 30 minutes to about 8 hours, from about 60 minutes to about 8 hours, from about 20 minutes to about 6 hours, from about 20 minutes to about 3 hours, from about 20 minutes to about 2 hours, from about 45 minutes to about 3 hours, etc.), and then the CRISPR activity within the cell, such as cutting of the target nucleic acid, is measured using method known to those of skill in the art. For example, total nucleic acid can be isolated from cells to be tested for CRISPR system activity and then analyzed for the amount of nucleic acid that has been such at the target locus, such as by measuring mutation induction as a proxy for cutting at the target locus of the nucleic acid.

Delivery of System Components into Wells of a Well Plate

According to one aspect, one or more cells or components of a CRISPR/Cas system are delivered to wells of a well plate using methods known to those of skill in the art. According to one aspect, a microfluidics system is used where one or more conduits connected to one or more reservoirs including one or more reagents is provided such that for each well of a plurality of wells, a cell is transferred to a well and one or more components of a CRISPR/Cas system are transferred to the well. Systems are commercially available for the transport of fluids into wells of a multi- well plate such as a multidrop dispenser (Thermo Fisher Scientific). Once the cells and CRISPR components are transferred to the plurality of wells, the plurality of wells are incubated or otherwise placed under conditions where the components of the CRISPR/Cas system are provided into the cell, a colocalization complex forms between a guide RNA, a Cas enzyme and a target nucleic acid, and the Cas enzyme cuts the target nucleic acid and a mutated gene results.

According to one aspect, cells and/or components of a CRISPR/Cas system are delivered to wells of a well plate using acoustic droplet ejection of reagents from a source plate positioned below the well plate to a well of the well plate positioned above the source plate. Sound waves eject precisely sized droplets from a source into the well suspended above the source. Exemplary delivery systems are commercially available from LABCYTE and include the ECHO liquid handling technology. According to one aspect, one or more or all of a liposome comprising a crRNA portion of a guide RNA, a liposome comprising a tracrRNA portion of the guide RNA, a liposome comprising a Cas enzyme or a nucleic acid sequence encoding the Cas enzyme, and a target cell are provided to the well by droplet transfer from a source container to the well using sound waves.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example I

### General Transfection Protocol for CRISPR/Cas9 Reagents

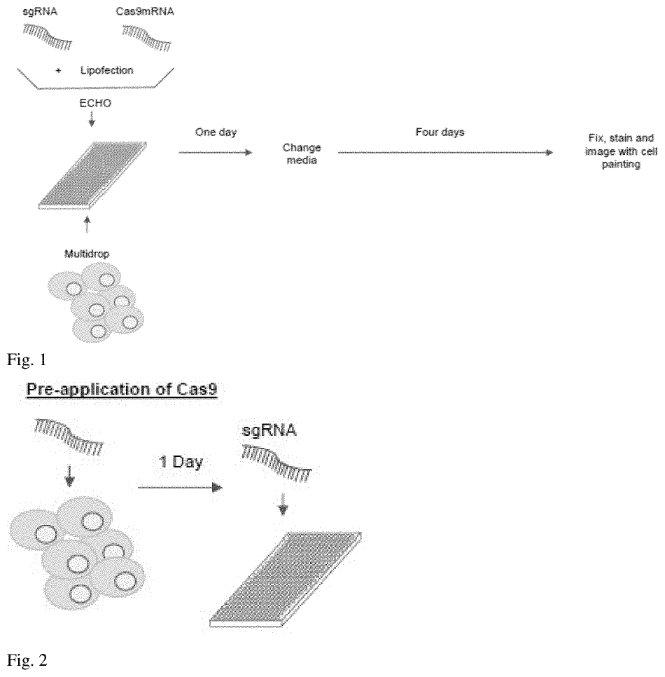

Cells are transfected as follows and with reference to the schematic shown in FIG. 1. Cas9mRNA and guide RNA complementary to a target nucleic acid is combined with a lipofection reagent to create a mixture of liposomes including the Cas9mRNA and the guide RNA (“reagent mixture”). The crRNA and the tracrRNA can be included into separate liposomes as separate reagents added to the mixture. Suitable lipofection reagents include RNAMAX, CRISPRMAX or LIPOFECTAMINE 2000, and the like. The reagent mixture is then delivered to wells of a multi- well plate using acoustic droplet ejection. According to this method, a fluid source including a reagent (or a cell) is operatively connected to an acoustic generator. A multi-well plate is positioned above the fluid source in an inverted position so that the wells are facing the fluid source. Sounds waves are generated causing droplets to be ejected from the fluid source up and into a well of the multi-well plate where they are retained despite the multi-well plate being inverted. A plurality of fluid sources may be positioned below the multi-well plate such that multiple wells are provided with reagent simultaneously. Alternatively, a different fluid source can be positioned underneath a specific well to deliver a particular reagent, such as a unique crRNA sequence to the well for targeting a specific gene of a target cell. In this manner, each well of a plurality of wells may contain a different, unique crRNA. In addition, different fluid sources can be positioned underneath a specific well to deliver two or more particular reagents, such as a two or more unique crRNA sequence to the well for targeting a different genes of a target cell. In addition, a different fluid source can be positioned underneath a specific well to deliver two or more particular reagents, such as two or more unique crRNA sequences in a mixture to the specific well for targeting two or more specific genes of a target cell. Accordingly, the disclosure contemplates delivering a single unique crRNA to a specific well, but also a plurality of unique crRNAs to a specific well to target a plurality of genes in the cell or cells in the single well. The well concentration of the guide RNA may be between 10 nm and 1 μmol, such as between 25 pmol and 125 pmol. The well concentration of the Cas enzyme is between 1 nM and 150 nM, such as between 60 nM and 80 nM. Cells are then added to each well including the reagent mixture using either the acoustic droplet ejection system or a multidrop dispenser (Thermo Fisher Scientific). The cells are allowed to incubate with the reagent mixture for 24 hours. The media is then changed and the cells are allowed to incubate for an additional 96 hours. The cells are then removed from the wells, fixed, stained and imaged.

### Example II

### General Transfection Protocol for CRISPR/Cas9 Reagents (Pre-Application of Cas9)

With reference to FIG. 2, cells are combined with Cas9mRNA in a lipofection reagent to add the Cas9 to the cells prior to contacting the cells with the guide RNA. The cells transfected to include Cas9 are then mixed with guide RNA in a lipofection reagent and the mixture is added to the wells of a multi-well plate using either a multidrop dispenser or acoustic droplet ejection. Alternatively, the cells transfected to include the Cas9 may be added to the wells, and then a guide RNA reagent treated with a lipofection reagent to create liposomes containing the guide RNA may be added to the wells. Suitable lipofection reagents include RNAMAX, CRISPRMAX or LIPOFECTAMINE 2000. The well concentration of the guide RNA may be between 10 nm and 1 μmol, such as between 25 pmol and 125 pmol. The cells are allowed to incubate with the reagent mixture for 24 hours. The media is then changed and the cells are allowed to incubate for an additional 96 hours. The cells are then removed from the wells, fixed, stained and imaged.

### Example III

### General Transfection Protocol for CRISPR/Cas9 Reagents (Different gRNA Per Well)

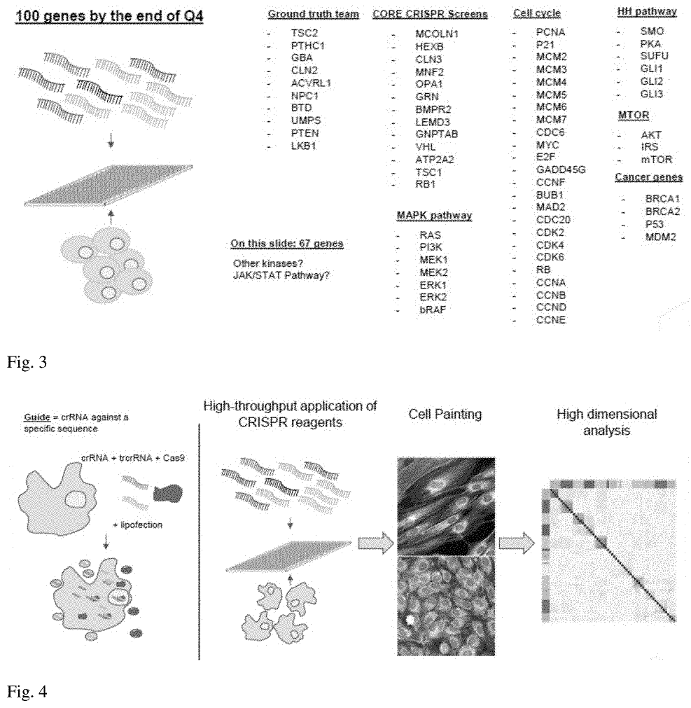

Cells are transfected as follows and with reference to the schematic shown in FIG. 3. Cells are delivered to wells of a 1536 multi-well plate using either a multidrop dispenser or using acoustic droplet ejection, for example. Cas9 mRNA is combined with a lipofection reagent to create liposomes including the Cas9 mRNA which is then added to the wells using either a multidrop dispenser or using acoustic droplet ejection, for example. Reagents, each of which including a different guide RNA complementary to a target nucleic acid, are made by combining each different guide RNA with a lipofection reagent to create liposomes including the guide RNA. Suitable lipofection reagents include RNAMAX, CRISPRMAX or LIPOFECTAMINE 2000. Each different guide RNA reagent is added to a different well using a multidrop dispenser or using acoustic droplet ejection. The well concentration of the guide RNA may be between 10 nm and 1 μmol, such as between 25 pmol and 125 pmol. The well concentration of the Cas enzyme is between 1 nM and 150 nM, such as between 60 nM and 80 nM. The cells are allowed to incubate with the reagent mixture for 24 hours. The media is then changed and the cells are allowed to incubate for an additional 96 hours. The cells are then removed from the wells, fixed, stained and imaged. Each well includes a cell with a different genomic modification.

Representative genes target by the guide RNA include those shown in FIG. 3.

### Example IV

### General Transfection Protocol for CRISPR/Cas9 Reagents (Combinations of Reagents)

According to aspects of the present disclosure, various reagents can be added at different times and in different orders using either a multidrop dispenser or using acoustic droplet ejection, for example. In general, methods described herein and with reference to FIG. 4 for genetically altering a plurality of target cells include the steps of (a) combining within each of a plurality of wells of a first well plate (1) a liposome comprising a crRNA portion of a guide RNA, wherein the crRNA portion includes a unique spacer complementary to a target gene, (2) a liposome comprising a tracrRNA portion of the guide RNA, (3) a liposome comprising a Cas enzyme or a nucleic acid sequence encoding the Cas enzyme, and (4) a target cell, and (b) incubating the plurality of target cells within the plurality of wells wherein the crRNA portion and the tracrRNA portion form the guide RNA and wherein the Cas enzyme and the guide RNA form a colocalization complex with the target gene of the target cell and the Cas enzyme cuts the target gene and mutates the target gene, wherein the unique spacer sequence of the crRNA portion in each well is complementary to a different target gene and wherein the location within the first well plate of each unique spacer sequence within each well is known, and the target cell within each well has a different target gene mutated. The process may be repeated for a given set of crRNAs but providing the crRNAs at different well locations, or the process may be carried out for a different set of crRNAs corresponding to a portion of the genome or the entire genome. In this manner, a high throughput method using CRISPR reagents is provided using an addressable array and a single knockout mutation per well at a genome scale. The cells may be analyzed, using cell painting and high dimensional analysis, for example.

According to one aspect, the liposome comprising the crRNA portion of a guide RNA, the liposome comprising the tracrRNA portion of the guide RNA, and the liposome comprising the Cas enzyme or a nucleic acid sequence encoding the Cas enzyme are provided to the well before the target cell is provided to the well. According to one aspect, the target cell is provided to the well before the liposome comprising the crRNA portion of a guide RNA, the liposome comprising the tracrRNA portion of the guide RNA, and the liposome comprising the Cas enzyme or a nucleic acid sequence encoding the Cas enzyme are provided to the well. According to one aspect, the liposome comprising the tracrRNA portion of the guide RNA, the liposome comprising the Cas enzyme or a nucleic acid sequence encoding the Cas enzyme, and the target cell are provided to the well before the liposome comprising the crRNA portion of a guide RNA is provided to the well. According to one aspect, the liposome comprising the crRNA portion of a guide RNA is provided to the well before the liposome comprising the tracrRNA portion of the guide RNA, the liposome comprising the Cas enzyme or a nucleic acid sequence encoding the Cas enzyme, and the target cell are provided to the well. According to one aspect, the liposome comprising the crRNA portion of a guide RNA is provided to the well after the liposome comprising the tracrRNA portion of the guide RNA, the liposome comprising the Cas enzyme or a nucleic acid sequence encoding the Cas enzyme, and the target cell are provided to the well. According to one aspect, the liposome comprising the tracrRNA portion of the guide RNA and the liposome comprising the Cas enzyme or a nucleic acid sequence encoding the Cas enzyme are provided to the well as a mixture. According to one aspect, the liposome comprising the tracrRNA portion of the guide RNA, the liposome comprising the Cas enzyme or a nucleic acid sequence encoding the Cas enzyme, and the target cell are provided to the well as a mixture. According to one aspect, the liposome comprising the Cas enzyme or a nucleic acid sequence encoding the Cas enzyme, and the target cell are provided to the well as a mixture. According to one aspect, the cell is transfected with the nucleic acid encoding the Cas enzyme before the target cell is provided to the well. According to one aspect, (1) the liposome comprising the crRNA portion of a guide RNA, (2) a mixture of the liposome comprising the tracrRNA portion of the guide RNA and the liposome comprising the Cas enzyme or a nucleic acid sequence encoding the Cas enzyme, and (3) the target cell are provided separately to the well. According to one aspect, the steps (a) and (b) above for the general method are repeated for a second well plate, and wherein each unique spacer sequence provided to the wells of the first well plate is provided at a location within the second well plate that is different from the first well plate. According to one aspect, the steps (a) and (b) above for the general method are repeated for a plurality of well plates, and wherein each unique spacer sequence provided to the wells of the first well plate is provided at a different location within each of the plurality of well plates and the first well plate.

According to one aspect, CRISPR activity with respect to each gene of an entire genome can be assayed or analyzed using high throughput genetic mutation of each gene. A crRNA is designed for each gene. Directing each crRNA to a single well and using a 1536 multi-well plate as described herein, the entire genome of a target cell can be assayed for CRISPR activity using about 1200 1536 multi-well plates. Such a whole genome assay can be carried out in about 3 weeks when processing about 400 plates per week.

According to one aspect, methods provided herein and discussed above allow for randomization of the CRISPR activity by generating data obtained from different well locations but directed to the cutting of a particular gene. In this manner, any influence or artifacts generated by well location can be minimized.

### Example V

### Specific Transfection Protocol for CRISPR/Cas9 Reagents

To prepare for the lipofection of cells, reagents are iteratively added to a 1536 well plate. First, crRNAs are dispensed to the plate using an Echo Acoustic Liquid Handler. 10 uL of one or multiple crRNAs are dispensed to each well, at a concentration of 200 uM. After dispensing, these plates are sealed and stored at 4° C. These plates should be stored at 4° C. for no more than one month.

According to the methods described herein, the lipofectamine mixture is prepared. Each 1536 well plate requires 3.2 mL of lipofectamine mixture. Table 1 and Table 2 below provide quantities for 1 mL. One of skill can adjust or ramp up amounts based on the size of a desired experiment. Two 15mL conical tubes are labeled “A” and “B”. Prepare tube A by combining the reagents listed in Table 1 in the order that they are listed, making sure vortexing after the addition of tracrRNA and optionally a component that aids Cas9 transfection into cells if needed, such as Cas9 PLUS commercially available from ThermoFisher. Rest the tube at room temperature for 5 minutes before using it in the next step.

TABLE 1
Volumes required for 1 mL
Tube A

| | |
|---|---|
| Optimem | 473.34 |
| Cas9 | 2.825 |
| tracrRNA | 3 |
| Cas9 Plus | 20.83 |

Prepare tube B by combining the reagents in the order listed in Table 2.

TABLE 2
Volumes required for 1 mL
Tube B

| | |
|---|---|
| Optimem | 493.75 |
| CRISPRMax lipofection | 6.25 |
| reagent | |

Allow this tube to rest at room temperature for five minutes before it is used in the next step. To complete the preparation of the lipofectamine mixture, pour the contents of tube A into tube B. Gently swirl them together until the mixtures are combined.

When the lipofectamine mixture is ready, add it to the 1536 well plate. Dispense 2 uL of mixture per well using an automated liquid handler. Following this, dispense 8 uL of cells at 125,000 cells/mL to each well using an automated liquid handler. After 1 hour, transfer the plates with lipofectamine and cells to an incubator at 37° C. After 24 hours, change the media on the plates to remove the lipofectamine mixture. This media change should use media appropriate for the cell type in question. Conduct a second media change after an additional 48 hours (or 72 hours into the experiment). After 96 hours, the samples are ready for downstream analysis.

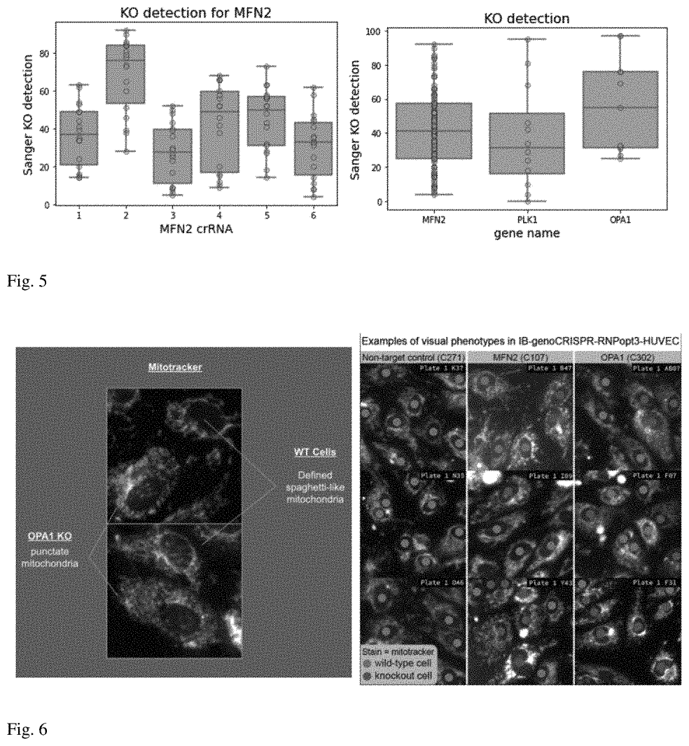

Gene knockout was validated for the above procedure. To confirm that cells were successfully edited using the method described herein, fluorescent images of cells which had been targeted for specific gene knockout were analyzed. In a first example, the gene editing rate was measured using Sanger sequencing of the cellular genomic DNA. This method can detect the proportion of edited cells using CRISPR to unedited cells in each sample. The first panel of FIG. 5 details the results of using six different CRISPR reagents against the gene MFN2 to generate edited cells, and demonstrates that the method can produce editing rates around 50%. The second panel of FIG. 5 contains results for the same procedure, but done for the genes PLK1 and OPAL Both panels demonstrate observable editing of cells.

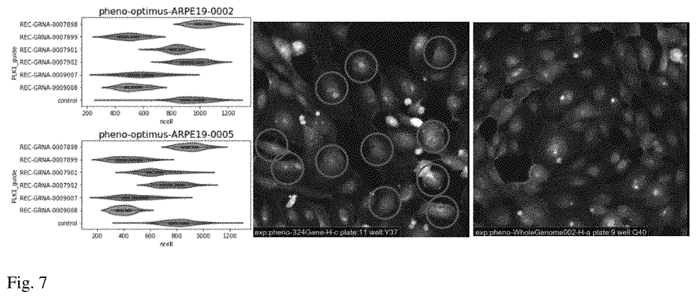

For additional confirmation of gene editing, the MFN2 and OPA1 genes were targeted for knockout, and cells were stained with a fluorescent dye to observe patterns of mitochondrial morphology. Both of these genes are required for regulating the stability of mitochondria in the cell—removing these genes causes mitochondria to fragment. This causes the mitochondria to go from a stringy, spaghetti like morphology to a punctate, dispersed morphology as exemplified in the first panel of FIG. 6. By analyzing sections of images from cells treated with MFN2, OPA1, or the non-targeting control reagents, wells that are targeted for MFN2 or OPA1 knockout contain the fragmented mitochondria phenotype.

In the second example, cells were targeted for deletion of PLK1, a protein that is involved in the cell cycle. Knockout of PLK1 leads to either cell death or multinucleated cells. The first panel of FIG. 7. shows the distribution of cell counts observed in a 1536 well plate when cells are treated with different reagents that target the PLK1 gene. As compared to control, four of the six PLK1 targeting reagents significantly reduce the observed cell count, which is the predicted effect of PLK1 knockout. Additionally, multinucleated cells are also observed in wells that are targeted by the cell count reducing PLK1 reagents. An example of this is provided in FIG. 7, where the multinucleated cells are circled in red. This phenotype is not observed in similar samples of unedited cells.

The above experiments demonstrate production of predictable, visible phenotypes in cultured cells.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of genetically altering a plurality of target cells comprising
(a) combining within each of a plurality of wells of a first well plate (1) a liposome comprising a crRNA portion of a guide RNA, wherein the crRNA portion includes a unique spacer complementary to a target gene, (2) a liposome comprising a tracrRNA portion of the guide RNA, (3) a liposome comprising a Cas enzyme or a nucleic acid sequence encoding the Cas enzyme, and (4) a target cell, and
(b) incubating the plurality of target cells within the plurality of wells wherein the crRNA portion and the tracrRNA portion form the guide RNA and wherein the Cas enzyme and the guide RNA form a colocalization complex with the target gene of the target cell and the Cas enzyme cuts the target gene and mutates the target gene,
wherein the unique spacer sequence of the crRNA portion in each well is complementary to a different target gene and wherein the location within the first well plate of each unique spacer sequence within each well is known, and the target cell within each well has a different target gene mutated.

2. The method of claim 1 wherein in step (a) at least two or more crRNA portions of a guide RNA, each with a unique spacer complementary to a different target gene, are separately provided within liposomes, and in step (b) at least two or more different target genes are knocked out in each target cell.

3. The method of claim 1 wherein the plurality of wells includes 500 or more wells.

4. The method of claim 1 wherein the plurality of wells includes 1000 or more wells.

5. The method of claim 1 wherein the plurality of wells includes 1536 or more wells.

6. The method of claim 1 wherein the plurality of wells includes 3000 or more wells.

7. The method of claim 1 wherein the first well plate includes edge wells and wherein the edge wells are vacant.

8. The method of claim 1 wherein each target cell within the plurality of target cells is a same cell type.

9. The method of claim 1 wherein each target cell within the plurality of target cells is a different cell type.

10. The method of claim 1 wherein the liposome comprising the crRNA portion of a guide RNA, the liposome comprising the tracrRNA portion of the guide RNA, and the liposome comprising the Cas enzyme or a nucleic acid sequence encoding the Cas enzyme are provided to the well before the target cell is provided to the well.

11. The method of claim 1 wherein the target cell is provided to the well before the liposome comprising the crRNA portion of a guide RNA, the liposome comprising the tracrRNA portion of the guide RNA, and the liposome comprising the Cas enzyme or a nucleic acid sequence encoding the Cas enzyme are provided to the well.

12. The method of claim 1 wherein the liposome comprising the tracrRNA portion of the guide RNA, the liposome comprising the Cas enzyme or a nucleic acid sequence encoding the Cas enzyme, and the target cell are provided to the well before the liposome comprising the crRNA portion of a guide RNA is provided to the well.

13. The method of claim 1 wherein the liposome comprising the crRNA portion of a guide RNA is provided to the well before the liposome comprising the tracrRNA portion of the guide RNA, the liposome comprising the Cas enzyme or a nucleic acid sequence encoding the Cas enzyme, and the target cell are provided to the well.

14. The method of claim 1 wherein the liposome comprising the crRNA portion of a guide RNA is provided to the well after the liposome comprising the tracrRNA portion of the guide RNA, the liposome comprising the Cas enzyme or a nucleic acid sequence encoding the Cas enzyme, and the target cell are provided to the well.

15. The method of claim 1 wherein the liposome comprising the tracrRNA portion of the guide RNA and the liposome comprising the Cas enzyme or a nucleic acid sequence encoding the Cas enzyme are provided to the well as a mixture.

16. The method of claim 1 wherein the liposome comprising the tracrRNA portion of the guide RNA, the liposome comprising the Cas enzyme or a nucleic acid sequence encoding the Cas enzyme, and the target cell are provided to the well as a mixture.

17. The method of claim 1 wherein the liposome comprising the Cas enzyme or a nucleic acid sequence encoding the Cas enzyme, and the target cell are provided to the well as a mixture.

18. The method of claim 1 wherein the cell is transfected with the nucleic acid encoding the Cas enzyme before the target cell is provided to the well.

19. The method of claim 1 wherein (1) the liposome comprising the crRNA portion of a guide RNA, (2) a mixture of the liposome comprising the tracrRNA portion of the guide RNA and the liposome comprising the Cas enzyme or a nucleic acid sequence encoding the Cas enzyme, and (3) the target cell are provided separately to the well.

20. The method of claim 1 wherein steps (a) and (b) are repeated for a second well plate, and wherein each unique spacer sequence provided to the wells of the first well plate is provided at a location within the second well plate that is different from the first well plate.

21. The method of claim 1 wherein steps (a) and (b) are repeated for a plurality of well plates, and wherein each unique spacer sequence provided to the wells of the first well plate is provided at a different location within each of the plurality of well plates and the first well plate.

22. The method of claim 21 wherein the plurality of well plates is greater than 1000 well plates.

23. The method of claim 1 wherein no more than one cell is present in each well.

24. The method of claim 1 wherein in step (a) the crRNA portion of a guide RNA and the tracrRNA portion of the guide RNA are linked and provided as a single guide RNA within a liposome.

25. The method of claim 1 wherein the well concentration of guide RNA is between 10 nM and 1 μM.

26. The method of claim 1 wherein the well concentration of guide RNA is between 25 pmol and 125 pmol.

27. The method of claim 1 wherein the well concentration of guide RNA is between 75 pmol and 125 pmol.

28. The method of claim 1 wherein the well concentration of Cas enzyme is between 1 nM and 150 nM.

29. The method of claim 1 wherein the well concentration of Cas enzyme is between 60 nM and 80 nM.

30. The method of claim 1 wherein the liposome comprising a crRNA portion of a guide RNA, the liposome comprising a tracrRNA portion of the guide RNA, the liposome comprising a Cas enzyme or a nucleic acid sequence encoding the Cas enzyme, and the target cell are provided to the well by droplet transfer from a source container to the well using sound waves.

31. The method of claim 1 wherein the Cas enzyme is a Cas enzyme of a Type II CRISPR system.
